SEQUENCE LISTING (2 sequences)

SEQ ID NO: 1 - DNA, 21 bp, Artificial Sequence
  Description of Artificial Sequence Primer
ggaagcagggagatagcagag

SEQ ID NO: 2 - DNA, 27 bp, Artificial Sequence
  Description of Artificial Sequence Primer
taggttgcaaagcacaaaatggagtca

(19) United States
(12) Patent Application Publication
(10) Pub. No.: US 2004/0063102 A1
(43) Pub. Date: Apr. 1, 2004

(54) Genetic marker and test

(76) Inventor: Roderick Clifton-Bligh, Glebe (AU)

Correspondence Address: Bennett J. Berson, Quarles & Brady LLP, 1 South Pinckney Street, P O Box 2113, Madison, WI, 53701-2113, US

(21) Appl. No.: 10/260,826

(22) Filed: Sep. 27, 2002

(51) Int. Cl. C12Q 1/68; C12P 19/34

(52) U.S. Cl. 435/6; 435/91.2

(57) ABSTRACT

A method of determining the susceptibility of a vertebrate to a bone-related disorder, the method comprising the steps of obtaining a DNA sample from the vertebrate; and analysing the DNA sample to determine whether the vertebrate has a polymorphism in the 1α-hydroxylase gene. Presence of the polymorphism indicates susceptibility of the vertebrate to the bone-related disorder.

### BACKGROUND OF THE INVENTION

The present invention relates to genetic markers and to methods of using such markers for assessing the predisposition of a subject to a disease state. More particularly the present invention relates to methods and kits for determining susceptibilty to bone-related disorders, such as osteoporosis and osteopenia.

Osteoporosis is a metabolic bone-related disorder characterized by low bone mass and deterioration of bone microarchitecture leading to increased risk of fracture. The importance of osteoporosis as a major public health burden has led to a global effort to understand its causes and possible prevention. Twin and pedigree studies have suggested that between 75-85% of the variance in bone mineral density is under genetic control. However a major difficulty has been in identifying polymorphisms which provide a reliable indicator of the predisposition of an individual to develop osteoporosis.

Polymorphic variants of several genes have been associated with variations in bone density, including the vitamin D receptor gene (VDR), the collagen 1α1 gene (Collα1), the oestrogen receptor a gene (ERα) and the PTH/PTHrP receptor gene (PTHR1). Each of these genes has been considered to be a plausible candidate because of the well-established physiological roles of their protein products as essential regulators of mineral ion homeostasis and bone development. For instance, the principal actions of vitamin D are to increase absorption of dietary calcium from the intestine, and to affect osteoblast and osteoclast function. However, the role of VDR in the pathogenesis of osteoporosis has been controversial, and an initial report showing that common VDR allelic variants predicted bone density has not consistently been supported by findings from other populations (Garnero et al., 1995).

Vitamin D itself is a biologically inert steroid made in the skin by the action of sunlight. The biologically active metabolite 1,25-dihydroxyvitamin D is formed from this precursor by successive 25-hydroxylation in the liver and 1α-hydroxylation in the kidney. The biologic activity of 1,25-dihydroxyvitamin D is mediated via its cognate vitamin D-receptor (VDR), a nuclear receptor that is present in all target tissues for vitamin D. The mitochondrial enzyme 1α-hydroxylase is critical to the synthesis of the active metabolite 1,25-dihydroxyvitamin D. The gene for 1α-hydroxylase in humans, CYP27B1 (alternatively termed P450c1α), has recently been cloned and shown to reside on chromosome 12, at 12q13.1-13.3, close to the VDR gene.

Impaired 1α-hydroxylase activity has been implicated in the development of osteoporosis, from observations that declining serum 1,25-dihydroxyvitamin D concentrations with age is accompanied by calcium malabsorption (Gallagher et al., 1979). However to date no direct association between 1α-hydroxylase and either osteoporosis, or reliable indicators thereof, has been found.

Accordingly, there is a need for the identification of reliable genetic markers for bone-related disorders including osteoporosis and for a method of determining the predisposition of an individual to such bone-related disorders using these markers.

The present invention is based on the unexpected finding that an association exists between a single base polymorphism in the 1α-hydroxylase gene and various physiological parameters which serve as indicators of the likelihood of development of bone-related disorders such as osteoporosis and osteopenia.

### SUMMARY OF THE INVENTION

According to a first embodiment of the present invention there is provided a method of determining the susceptibility of a vertebrate to a bone-related disorder, the method comprising the steps of:

(i) obtaining a DNA sample from the vertebrate; and

(ii) analysing the DNA sample to determine whether the vertebrate has a polymorphism in the 1α-hydroxylase gene, presence of the polymorphism indicating the susceptibility of the vertebrate to the bone-related disorder.

Typically the bone-related disorder is osteopenia or osteoporosis.

Typically, the polymorphism is in intron 6 of the 1α-hydroxylase gene. More typically, the polymorphism lies 29 base pairs from the start of exon 7.

Typically, the polymorphism is detected by the presence or absence of the sequence 5′ GTYRAC-3′ within intron 6. More typically, the sequence 5′-GTYRAC-3′ is detected by restriction enzyme analysis. Even more typically, the restriction enzyme used is HincII or HindII.

Typically, the analysis step (ii) involves:

(a) amplification of the DNA sample obtained from the vertebrate;

(b) restriction enzyme digestion of the amplified DNA using a restriction enzyme which recognises and cleaves the sequence 5′-GTYRAC-3′; and

(c) detection of the presence or absence of restriction enzyme cleavage of the DNA.

More typically the primers used in the amplification of the DNA sample are of the sequence 5′-GGA AGC AGG GAG ATA GCA GAG-3′ (SEQ ID NO:1) and 5′-TAG GTT GCA AAG CAC AAA ATG GAG TCA-3′ (SEQ ID NO:2). More typically, the presence or absence of restriction enzyme cleavage is determined by electrophoresis of the DNA.

Typically, the polymorphism in the 1α-hydroxylase gene is associated with altered bone mineral density.

According to a second embodiment of the present invention there is provided a method of diagnosing a bone-related disorder in a vertebrate, the method comprising the steps of:

(i) obtaining a DNA sample from the vertebrate; and

(ii) analysing the DNA sample to determine whether the vertebrate has a polymorphism in the 1α-hydroxylase gene, wherein presence of the polymorphism is an indicator of the bone-related disorder.

According to a third embodiment of the present invention there is provided a kit for use in determining the susceptibility of a vertebrate to a bone-related disorder, the kit comprising at least one primer designed to specifically amplify the 1α-hydroxylase gene and allow detection of the presence of a polymorphism in the 1α-hydroxylase gene, wherein the presence of the polymorphism indicates the susceptibility of the vertebrate to the bone-related disorder.

According to a fourth embodiment of the present invention there is provided a method of detecting a polymorphism in the 1α-hydroxylase gene of a vertebrate, the method comprising the steps of:

(i) obtaining a DNA sample from the vertebrate; and

(ii) analysing the DNA sample to determine the presence or absence of the sequence 5′-GTYRAC-3′ within intron 6 of the 1α-hydroxylase gene;

wherein the polymorphism is an indicator of the susceptibility of the vertebrate to a bone-related disorder.

According to a fifth embodiment of the present invention there is provided a kit for detecting a polymorphism in intron 6 of the 1α-hydroxylase gene of a vertebrate, the kit comprising at least one primer designed to specifically amplify the 1α-hydroxylase gene and allow detection of the polymorphism by the presence or absence of the sequence 5′-GTYRAC-3′ within intron 6, wherein the polymorphism is an indicator of the susceptibility of the vertebrate to a bone-related disorder. Typically, for the purposes of the present invention, the vertebrate is selected from the group consisting of human, non-human primate, murine, bovine, ovine, equine, caprine, leporine, avian, feline and canine. More typically, the vertebrate is human, non-human primate or murine. Even more typically, the vertebrate is human.

Typically, in each of the embodiments of the invention, the bone-related disorder is osteoporosis or osteopenia.

### BRIEF DESCRIPTION OF THE DRAWINGS

The present invention will now be described, by way of example only, with reference to the following drawings.

FIG. 1. Schematic representation of 1α-hydroxylase gene coding region showing exons (numbered boxes) and the position of the intronic T/C polymorphism (arrow) 29 bp upstream of the start of exon 7.

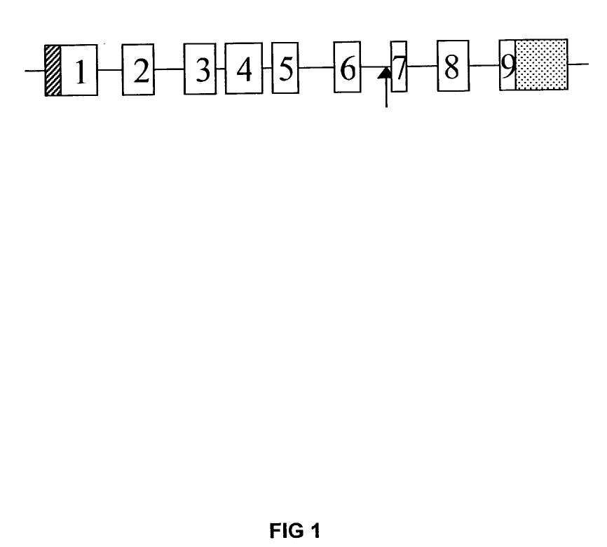

FIG. 2. Representative photograph of an agarose gel showing the resolution of 1α-hydroxylase genotypes TT, TC and CC. Sizes of nucleotide fragments are indicated on the left of the figure in base pairs (bp).

### Definitions

In the context of this specification, the term “comprising” means “including principally, but not necessarily solely”. Furthermore, variations of the word “comprising”, such as “comprise” and “comprises”, have correspondingly varied meanings.

The term “primer” as used herein means a single-stranded oligonucleotide capable of acting as a point of initiation of template-directed DNA synthesis. An “oligonucleotide” is a single-stranded nucleic acid typically ranging in length from 2 to about 500 bases. The precise length of a primer will vary according to the particular application, but typically ranges from 15 to 30 nucleotides. A primer need not reflect the exact sequence of the template but must be sufficiently complementary to hybridize to the template.

The term “intron” as used herein means a non-coding, intervening sequence within a gene that separates coding sequences (“exons”). Introns are removed from the message (RNA) and exons spliced together to determine the structure and sequence of the gene product. Introns thus do not contribute to the sequence of the gene product.

The term “genotype” as used herein means the genetic constitution of an organism. This may be considered in total, or as in the present application, with respect to the alleles of a single gene (that is, at a given genetic locus). Accordingly, the term “homozygote” refers to an organism that has identical alleles at a given locus on homologous chromosomes, whereas the term “heterozygote” refers to an organism in which different alleles are found on homologous alleles for a given locus.

The term “base pair” as used herein means a pair of nitrogenous bases, each in a separate nucleotide, in which each base is present on a separate strand of DNA and the bonding of these bases joins the component DNA strands. Typically a DNA molecule contains four bases; A (adenine), G (guanine), C (cytosine), and T (thymidine). A and G are purine bases, typically designated by the letter “R”, whereas C and T are pyrimidine bases, typically designated by the letter “Y”. The term “base pair” is abbreviated to “bp”.

The term “restriction enzyme” as used herein means an endonuclease enzyme that recognises and cleaves a specific sequence of DNA (recognition sequence).

### BEST MODE OF PERFORMING THE INVENTION

A polymorphism has recently been reported in intron 6 of the 1α-hydroxylase gene, 29 base pairs 5′ to the start of exon 7 (IVS-29 bp) (Smith et al., 1999) at position 3245 of the 1 a-hydroxylase gene sequence (GenBank Accession No. AF027152; Fu et al., 1997). This polymorphism is a single nucleotide polymorphism, the polymorphic residue being a C or T base. Specifically, the polymorphism is located within the sequence 5′-GTTGTC-3′ (the polymorphic T residue is indicated in bold). In the present invention a novel PCR/restriction analysis-based strategy has been developed to assign alleles of the 1α-hydroxylase gene based on this polymorphism and the association between this polymorphism and the physiological parameters of lumbar spine bone mineral density and serum levels of parathyroid hormone (PTH). A primer alters the sequence 5′-GTTGTC-3′, containing the polymorphism, to 5′-GTTGAC-3′ during PCR amplification. As a result, in the presence of the polymorphic T residue, the sequence is cleaved by a restriction enzyme which has as its recognition sequence the degenerate sequence 5′-GTYRAC-3′ (where Y=C or T; and R+A or G). In particular, this sequence is recognised and cleaved by the restriction enzyme HincII (HindII).

Alternatively, if a C residue is present at position 3245 (IVS-29 bp) in place of the T residue shown above, the genomic sequence becomes 5′-GCTGTC-3′. During PCR amplification this sequence will be altered to 5′-GCTGAC-3′, which will not be recognised, and therefore will not be cleaved by the HincII restriction enzyme.

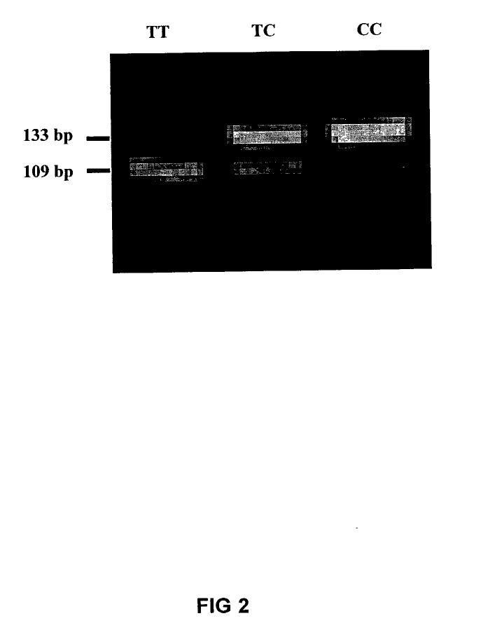

A representative image of the different alleles of the 1α-hydroxylase gene resolved by agarose gel electrophoresis is shown in FIG. 2. Based on this T/C polymorphism, three genotypes are possible; the homozygote genotypes TT and CC and the heterozygote genotype TC.

The present invention is based on the surprising finding that the 1 a-hydroxylase genotype of an individual, with respect to the T/C polymorphism (IVS-29 bp), correlates with various physiological parameters, in particular, lumbar spine bone mineral density (BMD) and serum levels of PTH. The TT 1α-hydroxylase genotype is associated with lower lumbar spine bone density and higher concentrations of serum PTH compared to the CC genotype, with an intermediate value in each case for the heterozygous TC genotype. These findings suggest that the 1α-hydroxylase genotype may be used as an indicator of the bone-related disorders osteoporosis and osteopenia.

Accordingly, the present invention provides methods and kits for the for the determination of the susceptibility of a subject to develop osteoporosis and/or osteopenia based on the presence or absence of the T/C polymorphism. The present invention additionally provides methods and kits for the detection of the T/C polymorphism in the 1α-hydroxylase gene.

### Methods

According to the present invention, a method particularly suitable for the detection of a polymorphism in the 1α-hydroxylase gene and for determining the susceptibility of a subject, is a PCR/restriction enzyme analysis strategy as described above.

DNA from the subject to be assessed may be extracted by a number of suitable methods known to those skilled in the art, for example salting out. Most typically, DNA is extracted from a blood sample, and in particular from peripheral blood leucocytes.

The methods and reagents for use in a PCR amplification reaction are well known to those skilled in the art. Suitable protocols and reagents will largely depend on individual circumstances. Guidance may be obtained from a variety of sources, such as for example Sambrook et al., Molecular Cloning: A Laboratory Manual, Cold Spring Harbor, N.Y., 1989, and Ausubel et al., Current Protocols in Molecular Biology, Greene Publ. Assoc. and Wiley-Intersciences, 1992. A typical PCR reaction (25 μL volume) contains 0.2 μM each forward (5′-GGA AGC AGG GAG ATA GCA GAG-3′) (SEQ ID NO:1) and reverse (5′-TAG GTT GCA AAG CAC AAA ATG GAG TCA-3′) (SEQ ID NO:2) oligonucleotide primers, 0.5 μg genomic DNA, 1.25 mM each dNTP (dATP, dCTP, dTTP, dGTP), 2.5 mM MgCl₂, 50 mM KCl, 10 mM Tris-HCl, and 0.5 unit of Taq polymerase. After an initial 2 minute denaturation at 94° C., reactions continue for 35 cycles of 94° C. for 30 s, 60° C. for 30 s and 72° C. for 60 s in a DNA engine thermal cycler. A person skilled in the art would readily appreciate that various parameters of the PCR reaction may be altered without affecting the ability to amplify the desired product. For example the Mg²⁺ concentration may be varied. Similarly, the amount of genomic DNA used a template may also be varied depending on the amount of DNA available.

The primers of the present invention are typically oligonucleotides of, generally, 15 to 20 bases in length. Such primers can be prepared by any suitable method, including, for example, direct chemical synthesis or cloning and restriction of appropriate sequences. Not all bases in the primer need reflect the sequence of the template molecule to which the primer will hybridize, the primer need only contain sufficient complementary bases to enable the primer to hybridize to the template. The primer may include additional bases, for example in the form of a restriction enzyme recognition sequence at the 5′ end, to facilitate cloning of the amplified DNA. A primer may also include mismatch bases at one or more positions, being bases that are not complementary to bases in the template, but rather are designed to incorporate changes into the DNA upon amplification.

Suitable methods of analysis of the amplified DNA to determine the presence or absence of the polymorphism are well known to those skilled in the art. Following digestion of the amplified DNA with a suitable restriction enzyme, the DNA may be analysed by a range of suitable methods, including electrophoresis. Of particular use is agarose gel electrophoresis, a technique commonly used by those skilled in the art for separation of DNA fragments on the basis of size. The concentration of agarose in the gel in large part determines the resolution ability of the gel and the appropriate concentration of agarose will therefore depend on the size of the DNA fragments to be distinguished. In a preferred method according to the present invention, the restriction enzyme HincII is used to digest amplified DNA and determine the 1α-hydroxylase genotype. This requires the resolution of DNA fragments of 133 bp and 109 bp, which is effectively achieved on a 2.2% agarose gel.

### Kits

Kits for determining susceptibility to a disease state, and for detecting a polymorphism in the 1α-hydroxylase gene are also provided. Such kits can be used with the methods described herein. Typically, kits according to the present invention are designed specifically to enable the amplification and analysis of a segment of the 1α-hydroxylase gene containing intron 6, and in particular the position IVS-29 bp.

Accordingly, kits of the present invention typically include one or more primers that specifically hybridize to the 1α-hydroxylase gene. In such kits, appropriate amounts of the one or more primers are provided in suitable containers. The oligonucleotide primers may be provided suspended in an aqueous solution or as a freeze-dried or lyophilized powder, for example.

Typically a kit of the present invention includes two primers, at least one of which is designed to encompass a polymorphic site, and in particular wherein one primer introduces a specific restriction enzyme site with the sequence 5′-GTYRAC-3′, as described above. The appropriate sequences of the primers may vary, but primers of sequence 5′-GGA AGC AGG GAG ATA GCA GAG-3′ (SEQ ID NO:1) and 5′-TAG GTT GCA AAG CAC AAA ATG GAG TCA-3′ (SEQ ID NO:2) are particularly suitable. The amount of each primer supplied in the kit can be any appropriate amount, depending on the nature of the application, and would likely be an amount sufficient to prime at least several amplification reactions. A person skilled in the art would readily appreciate the appropriate amount of each primer to use in a single amplification reaction.

A kit according to the present invention may also include a suitable control template molecule and/or control primers for use in a control reaction. The design of suitable control templates and primers and of control reactions is well known to those skilled in the art.

A kit according to the present invention may additionally include other components for performing amplification reactions including, for example, DNA sample preparation reagents, appropriate buffers (e.g. polymerase buffer), salts (e.g. magnesium chloride), and deoxyribonucleotides (dNTPs). The kit may further include the necessary reagents for carrying out analysis of the amplified DNA, such as an appropriate restriction enzyme, reaction buffer for restriction enzyme digestion, and reagents for use in separating digested fragments (e.g. agarose). Typically, a kit may also include containers for housing the various components and instructions for using the kit components to conduct amplification reactions according to the present invention.

The present invention will now be further described in greater detail by reference to the following specific examples, which should not be construed as in any way limiting the scope of the invention.

### EXAMPLES

### Example 1

### 1α-Hydroxylase Gene Polymorphisms

Healthy Caucasian, women who were between 1 and 10 years post-menopause (age range 45 to 68 years) and not using hormone replacement therapy were recruited through media advertisements. Study participants were assessed by means of a medical history questionnaire and individuals with malignant disease, renal, hepatic, endocrine or gastrointestinal disorder associated with abnormal calcium or bone metabolism were excluded. Subjects who had used oestrogen, progesterone, glucocorticoids, anti-convulsants, thiazide diuretics, vitamin D supplements or other medication known to affect calcium or bone metabolism within 12 months of study entry were also excluded. Subjects with laboratory evidence of renal, hepatic or endocrine disorder, serum FSH <40 pmol/L or BMD at any site below 2 SD from the mean for subjects matched for age were also excluded. Of the 187 women who met all entry criteria and were enrolled in the study, 123 gave written informed consent for genetic analyses to be performed on genomic DNA extracted from their peripheral blood leucocytes.

Genomic DNA was extracted from peripheral blood leucocytes using standard procedures. The T/C polymorphism within intron 6 (29 base pairs from the start of exon 7) of the 1α-hydroxylase gene was detected by polymerase chain reaction (PCR) with a mismatched primer that introduces a HincII restriction site in the presence of the T allele. PCR reactions (25 μL volume) contained 0.2 μM each forward (5′-GGA AGC AGG GAG ATA GCA GAG-3′) (SEQ ID NO:1) and reverse (5′-TAG GTT GCA AAG CAC AAA ATG GAG TCA-3′) (SEQ ID NO:2) oligonucleotide primers, 0.5 μg genomic DNA, 1.25 mM each dNTP, 2.5 mM MgCl₂, 50 mM KCl, 10 mM Tris-HCl, and 0.5 unit of Taq polymerase (Perkin-Elmer, California). After an initial 2 minute denaturation at 94° C., reactions continued for 35 cycles of 94° C. for 30 seconds, 60° C. for 30 seconds and 72° C. for 60 seconds in a DNA engine thermal cycler (Geneworks, South Australia). The PCR products were digested at 37° C. overnight with 5 units HincII (Progen, Queensland) and resolved on a 2.2% agarose gel.

A representative image of the different alleles of the 1α-hydroxylase gene from the study population of 123 postmenopausal women, as resolved by agarose gel electrophoresis is shown in FIG. 2. The 1α-hydroxylase allele frequencies in our study population were 64% and 36% for the T and C alleles respectively. This is similar to those frequencies reported by Smith et al. (1999). The observed genotype distribution was 41% TT: 47% TC: 12% CC and did not differ from Hardy-Weinberg equilibrium (χ²=0.12, p>0.975).

### Example 2

### Correlations Between Various Factors and 1α-Hydroxylase Genotype

Height and weight of the women in the study population were measured at the initial examination, with the subject standing without shoes. Areal bone mineral density (BMD, g/cm²) was measured with dual-energy X-ray absorptiometer (XR26, Norland). External and internal calibrations were performed daily using a hydroxyapatite phantom in perspex. Serum 25 hydroxyvitamin D, 1,25-dihydroxyvitamin D and osteocalcin were measured by radioimmunoassay (Incstar Corp., Minnesota). Serum PTH was measured by in-house radioimmunoassay using antibodies against the intact molecule (Kleerekoper et al., 1974). Urinary concentrations of deoxypyridinoline crosslinks in fasting second morning voids were measured by competitive enzyme immunoassay (Metra Biosystems Inc., California). Dietary calcium was assessed at study baseline by means of a food assessment questionnaire (Angus et al., 1989).

Bone mineral density and other clinical variables from the three genotype groups were compared by analysis of co-variance, and data from the TT and CC genotype groups were compared by Student's t-test. Multi-variate linear regression was used to determine if 1α-hydroxylase genotype (assigned value of 0, 1 or 2 according to the number of C alleles present) was independently associated with either serum PTH concentrations or lumbar spine BMD after accounting for age, weight, height, dietary calcium intake, and VDR genotype. The chi-square test was used to test for Hardy-Weinberg equilibrium. All statistical tests were two-sided.

The baseline characteristics of the study population according to 1α-hydroxylase genotype are presented in Table 1. The average age of participants was 56.116.3 years (range 45-65 years) and did not differ significantly by 1α-hydroxylase genotype. In contrast, 1α-hydroxylase genotype was significantly associated with current height and weight, serum PTH and lumbar spine BMD Z-scores as measured at study baseline. It was found that women with the 1α-hydroxylase TT genotype had mean serum PTH concentration about 0.5 SDs higher than women with the CC genotype (0.25±0.30 pg/mL vs 0.15±0.08 pg/mL, p=0.04). Furthermore, the mean lumbar spine BMD Z-score was 0.5 SDs higher in women with the TT genotype compared with those with the CC genotype (−0.05±0.94 vs 0.53±1.05, p=0.05).

Intriguingly, women with the 1α-hydroxylase TT genotype were also significantly taller (163.5±5.9 vs 159.3±6.2 cm, p=0.03) and heavier (70.2±13.7 vs 63.7±8.5, p=0.03) at study baseline than those with the CC genotype. Weight and height are known to positively correlate with bone density (Langdahl et al., 2000). Therefore, when age, height, weight and 1α-hydroxylase genotype were considered together in a multivariate regression model using lumbar spine BMD (g/cm²) as the dependent variable, the association between lumbar spine BMD and 1α-hydroxylase genotype became more significant (p=0.014, Table 2). The 1α-hydroxylase genotype was not significantly associated with serum calcium, phosphate, 25-hydroxyvitamin D or 1,25-dihydroxyvitamin D concentrations, nor with BMD at femoral neck or distal radius and ulna (Table 1).

Table 1. Characteristics of 123 Postmenopausal Women According to Their 1α-Hydroxylase Genotype

TABLE 1
Characteristics of 123 postmenopausal woman according to their 1α-hydroxylase genotype

| | 1α-hydroxylase genotype | | | P value | |
|---|---|---|---|---|---|
| Characteristic | TT(n = 50) | TC(n = 58) | CC(n = 15) | ANOVA | CC vs TT |
| Demographic | | | | | |
| Age (yr) | 56.4 ± 4.4 | 55.8 ± 7.8 | 56.9 ± 4.9 | 0.79 | 0.73 |
| Years postmenopause | 6.2 ± 2.9 | 5.3 ± 2.8 | 6.4 ± 3.3 | 0.2 | 0.82 |
| Height (m) | 163.5 ± 5.9 | 162.7 ± 5.5 | 159.3 ± 6.2 | 0.05 | 0.03 |
| Weight (kg) | 70.2 ± 13.7 | 64.5 ± 9.8 | 63.7 ± 8.5 | 0.02 | 0.03 |
| Dietary calcium intake | 764 ± 280 | 771 ± 312 | 908 ± 333 | 0.14 | 0.24 |
| Past Smoking History (%) | 4 | 5 | 7 | 0.91 | 0.72 |
| Biochemistry | | | | | |
| Serum 1-84 PTH (pg/mL) | 0.25 ± 0.30 | 0.19 ± 0.07 | 0.15 ± 0.08 | 0.14 | 0.04 |
| Serum calcium (mmol/L) | 2.40 ± 0.09 | 2.40 ± 0.08 | 2.39 ± 0.06 | 0.91 | 0.61 |
| Serum 25 (OH) D (nmol/L) | 85 ± 26 | 87 ± 29 | 81 ± 18 | 0.97 | 0.80 |
| Serum 1, 25 (OH) | 93 ± 31 | 89 ± 34 | 88 ± 22 | 0.83 | 0.88 |
| D (pmol/L) | | | | | |
| Serum ALP (U/L) | 84 ± 21 | 87 ± 26 | 90 ± 16 | 0.65 | 0.30 |
| Serum Osteocalcin (ng/mL) | 4.5 ± 2.0 | 4.7 ± 2.3 | 3.8 ± 1.5 | 0.33 | 0.33 |
| Urine Dpyr | 4.8 ± 3.9 | 4.5 ± 2.1 | 4.1 ± 1.5 | 0.85 | 0.52 |
| (nmol/mmol/Cr) | | | | | |
| Bone mineral density | | | | | |
| Lumbar Spine (g/cm²) | 0.94 ± 0.15 | 0.96 ± 0.15 | 1.03 ± 0.17 | 0.16 | 0.09 |
| Lumbar Spine (Z-score) | −0.05 ± 0.94 | 0.05 ± 0.91 | 0.53 ± 1.05 | 0.12 | 0.05 |
| Femoral Neck (g/cm²) | 0.82 ± 0.12 | 0.80 ± 0.10 | 0.84 ± 0.14 | 0.44 | 0.64 |
| Femoral neck (Z-score) | −0.29 ± 1.02 | −0.45 ± 0.76 | 0.00 ± 1.09 | 0.24 | 0.38 |
| Distal radius + ulna (g/cm²) | 0.28 ± 0.06 | 0.29 ± 0.05 | 0.30 ± 0.08 | 0.26 | 0.35 |

TABLE 2
Multiple linear regression analysis

| | P value |
|---|---|
| Independent predictors of lumbar spine BMD | |
| Weight (kg) | 0.001 |
| years since menopause | 0.004 |
| 1α-hydroxylase genotype | 0.014 |
| Independent predictors of PTH | |
| 1α-hydroxylase genotype | 0.024 |
| Dietary calcium (g/day) | 0.034 |

### Example 3

### VDR Genotypes

The distribution of vitamin D receptor (VDR) genotypes for the BsmI allele in the study population from Example 1 were determined. Genotype assignment for the BsmI polymorphism in the VDR gene was determined as previously described (Langdahl et al., 2000). The distribution of VDR versus 1α-hydroxylase-genotypes did not differ significantly from that expected (Table 3). Individually, VDR genotype was not significantly associated with age, height, weight, serum PTH, or BMD at lumbar spine or femoral neck (Table 4). In the multivariate regression model using lumbar spine BMD as the dependent variable, VDR genotype did not interact with 1α-hydroxylase genotype (data not shown).

TABLE 3
Distribution of VDR BsmI and 1α-hydroxylase genotypes

| | 1α-hydroxylase genotype | | |
|---|---|---|---|
| VDR genotype | CC | TC | TT |
| bb | 8 | 18 | 24 |
| Bb | 7 | 30 | 22 |
| BB | 0 | 10 | 4 |
| | | | X²P = 0.18 |

Table 4. Characteristics of 123 Postmenopausal Women According to Their VDR BsmI Genotype

TABLE 4
Characteristics of 123 postmenopausal women according to their VDR BsmI genotype

| | VDR genotype | | | P value | |
|---|---|---|---|---|---|
| Characteristic | bb(n = 50) | Bb(n = 58) | BB(n = 15) | ANOVA | bb vs BB |
| Age (yr) | 56.6 ± 3.5 | 56.8 ± 5.1 | 55.6 ± 3.7 | 0.66 | 0.38 |
| Height (in) | 162.5 ± 6.1 | 162.3 ± 5.9 | 163.5 ± 5.5 | 0.80 | 0.57 |
| Weight(kg) | 66.5 ± 11.1 | 67 ± 11.2 | 65.5 ± 15.3 | 0.89 | 0.82 |
| Serum 1-84 PTH (pg/mL) | 0.23 ± 0.24 | 0.20 ± 0.18 | 0.19 ± 0.06 | 0.70 | 0.33 |
| Lumbar Spine (g/cm²) | 0.97 ± 0.15 | 0.95 ± 0.16 | 0.98 ± 0.13 | 0.69 | 0.74 |
| Femoral Neck (g/cm²) | 0.83 ± 0.11 | 0.81 ± 0.12 | 0.80 ± 0.11 | 0.67 | 0.41 |

### REFERENCES

Each of the following references is incorporated by reference as if set forth herein in its entirety.

Angus R M, Sambrook P N, Pocock N A, Eisman JA 1989. A simple method for assessing calcium intake in Caucasian women. J Am Diet Assoc 2:209-214.

Fu G K, Portale A A, Miller W L 1997. Complete structure of the human gene for the vitamin D 1alpha-hydroxylase, P450c 1alpha. DNA Cell Biol 16:1499-1507.

Gallagher J C, Riggs B L, Eisman J, Hamstra A, Arnaud S B, DeLuca H F 1979. Intestinal calcium absorption and serum vitamin D metabolites in normal subjects and osteoporotic patients: effect of age and dietary calcium. J Clin Invest 64:729-736.

Garnero P; Borel O; Sornay-Rendu E, Delmas P D 1995. Vitamin D receptor gene polymorphisms do not predict bone turnover and bone mass in healthy premenopausal women. J Bone Miner Res 10:1283-1288.

Kleerekoper M, Ingham J P, McCarthy S W, Posen S 1974. Parathyroid hormone assay in primary hyperparathyroidism: experiences with a radioimmunoassay based on commercially available reagents. Clin Chem 20:369-375.

Langdahl B L, Gravholt C H, Brixen K, Eriksen E F 2000. Polymorphisms in the vitamin D receptor gene and bone mass, bone turnover and osteoporotic fractures. Eur J Clin Invest 30:608-617.

Smith S J, Rucka A K, Berry J L, Davies M, Mylchreest S, Paterson C R, Heath D A, Tassabehji M, Read A P, Mee A P, Mawer E B 1999. Novel mutations in the 1alpha-hydroxylase (P450c1) gene in three families with pseudovitamin D-deficiency rickets resulting in loss of functional enzyme activity in blood-derived macrophages. J Bone Miner Res 14:730-739.

1. A method of determining the susceptibility of a vertebrate to a bone-related disorder, the method comprising the steps of:
(i) obtaining a DNA sample from the vertebrate; and
(ii) analysing the DNA sample to determine whether the vertebrate has a polymorphism in the 1α-hydroxylase gene, wherein presence of the polymorphism indicates susceptibility of the vertebrate to the bone-related disorder.

2. The method of claim 1, wherein the polymorphism is associated with altered bone mineral density in the vertebrate.

3. The method of claim 1, wherein the polymorphism resides in intron 6 of the 1α-hydroxylase gene.

4. The method of claim 3, wherein the polymorphism is a single nucleotide polymorphism, the polymorphic nucleotide being a cytosine or thymidine base located 29 bases from the start of exon 7 of the 1α-hydroxylase gene.

5. The method of claim 4, wherein analysis of the DNA sample includes the steps of:
(a) amplification of the DNA sample obtained from the vertebrate;
(b) digestion of the amplified DNA using a restriction endonuclease which recognises the sequence 5′-GTYRAC-3′; and
(c) detection of the presence or absence of restriction endonuclease cleavage of the digested DNA.

6. The method of claim 5, wherein the restriction endonuclease is HincII or HinduI.

7. The method of claim 5, wherein amplification of the DNA sample is carried out using one or more of the following primer sequences: 5′-GGA AGC AGG GAG ATA GCA GAG-3′ or 5′-TAG GTT GCA AAG CAC AAA ATG GAG TCA-3′.

8. The method of claim 1 wherein the bone-related disorder is osteoporosis or osteopenia.

9. The method of claim 1, wherein the vertebrate is selected from the group consisting of human, non-human primate, murine, bovine, ovine, equine, caprine, leporine, avian, feline and canine.

10. A method of diagnosing a bone-related disorder in a vertebrate, the method comprising the steps of:
(i) obtaining a DNA sample from the vertebrate; and
(ii) analysing the DNA sample to determine whether the vertebrate has a polymorphism in the 1α-hydroxylase gene, wherein presence of the polymorphism is an indicator of the bone-related disorder.

11. The method of claim 10, wherein the polymorphism is associated with altered bone mineral density in the vertebrate.

12. The method of claim 10, wherein the polymorphism resides in intron 6 of the 1α-hydroxylase gene.

13. The method of claim 12, wherein the polymorphism is a single nucleotide polymorphism, the polymorphic nucleotide being a cytosine or thymidine base located 29 bases from the start of exon 7 of the 1α-hydroxylase gene.

14. The method of claim 13, wherein analysis of the DNA sample includes the steps of:
(a) amplification of the DNA sample obtained from the vertebrate;
(b) digestion of the amplified DNA using a restriction endonuclease which recognises the sequence 5′-GTYRAC-3′; and
(c) detection of the presence or absence of restriction endonuclease cleavage of the digested DNA.

15. The method of claim 14, wherein the restriction endonuclease is HincII or HinduI.

16. The method of claim 14, wherein amplification of the DNA sample is carried out using one or more of the following primer sequences: 5′-GGA AGC AGG GAG ATA GCA GAG-3′ or 5′-TAG GTT GCA AAG CAC AAA ATG GAG TCA-3′.

17. The method of claim 10, wherein the bone-related disorder is osteoporosis or osteopenia.

18. The method of claim 10, wherein the vertebrate is selected from the group consisting of human, non-human primate, murine, bovine, ovine, equine, caprine, leporine, avian, feline and canine.

19. A kit for use in determining the susceptibility of a vertebrate to a bone-related disorder, the kit comprising at least one primer designed to specifically amplify the 1α-hydroxylase gene from the vertebrate and enable detection of the presence of a polymorphism in the 1α-hydroxylase gene, wherein the presence of the polymorphism indicates susceptibility of the vertebrate to the bone-related disorder.

20. The kit of claim 19 wherein the polymorphism is a single nucleotide polymorphism, the polymorphic nucleotide being a cytosine or thymidine base occurring 29 bases from the start of exon 7 of the 1α-hydroxylase gene.

21. The kit of claim 19, wherein the at least one primer comprises the sequence 5′-GGA AGC AGG GAG ATA GCA GAG-3′ or 5′-TAG GTT GCA AAG CAC AAA ATG GAG TCA-3′.

22. The kit of claim 19, wherein the kit includes two primers comprising the sequences 5′-GGA AGC AGG GAG ATA GCA GAG-3′ and 5′-TAG GTT GCA AAG CAC AAA ATG GAG TCA-3′.

23. The kit of claim 19, wherein the polymorphism is associated with altered bone mineral density in the vertebrate.

24. The kit of claim 19, wherein the bone-related disorder is osteoporosis or osteopenia.

25. The kit of claim 19, wherein the vertebrate is selected from the group consisting of human, non-human primate, murine, bovine, ovine, equine, caprine, leporine, avian, feline and canine.
